(19) United States
(12) Patent Application Publication
(10) Pub. No.: US 2024/0164784 A1
(43) Pub. Date: May 23, 2024

(54) LEFT ATRIAL APPENDAGE CLOSURE DEVICE WITH PETAL DESIGNS

(71) Applicant: BOSTON SCIENTIFIC SCIMED, INC., MAPLE GROVE, MN (US)

(72) Inventors: James M. Anderson, Corcoran, MN (US); Joshua Mark Inouye, Brooklyn Park, MN (US); Jerry Wang, Edina, MN (US)

(73) Assignee: BOSTON SCIENTIFIC SCIMED, INC., MAPLE GROVE, MN (US)

(21) Appl. No.: 18/516,028

(22) Filed: Nov. 21, 2023

Related U.S. Application Data
(60) Provisional application No. 63/427,659, filed on Nov. 23, 2022.

(51) Int. Cl. A61B 17/12; A61B 17/00

(52) U.S. Cl. CPC A61B 17/12122; A61B 17/0057; A61B 17/12168; A61B 2017/00632

(57) ABSTRACT

An implantable medical device such as, but not limited to an LAAC (left atrial appendage closure) device includes an expandable frame that is moveable between a collapsed configuration for delivery and an expanded configuration for deployment, the expandable frame having a variable diameter in the expanded configuration. The expandable frame includes a central member, and a grouping of arcuate articulating members joined at one end to the first central member, each of the first grouping of arcuate articulating members extending outwardly from the first central member. A covering is disposed relative to the first grouping of arcuate articulating members.

### CROSS REFERENCE TO RELATED APPLICATIONS

This application claims the benefit of priority of U.S. Provisional Application No. 63/427,659 filed Nov. 23, 2022, the entire disclosure of which is hereby incorporated by reference.

### TECHNICAL FIELD

The disclosure relates generally to medical devices and more particularly to medical devices that are adapted for closing off a left atrial appendage.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in an implantable medical device. The implantable medical device includes an expandable frame moveable between a collapsed configuration for delivery and an expanded configuration for deployment, the expandable frame having a variable diameter in the expanded configuration. The expandable frame includes a first central member, and a first grouping of arcuate articulating members joined at one end to the first central member, each of the first grouping of arcuate articulating members extending outwardly from the first central member. The implantable medical device includes a covering disposed relative to the first grouping of arcuate articulating members.

Alternatively or additionally, the expandable frame may be biased into the collapsed configuration for delivery, and is moved into the expanded configuration for deployment by rotating the first central member.

Alternatively or additionally, the expandable frame may be biased into the expanded configuration for deployment, and is moved into the expanded configuration by removing a constraint that holds the expandable frame in the collapsed configuration for delivery.

Alternatively or additionally, the covering may extend over the first grouping of arcuate articulating members.

Alternatively or additionally, the first grouping of arcuate articulating members may be formed of a shape memory material.

Alternatively or additionally, the first grouping of arcuate articulating members may be adapted to have a remembered shape corresponding to the expanded configuration of the expandable frame.

Alternatively or additionally, the expandable frame may further include a second central member, and a second grouping of arcuate articulating members joined at one end to the second central member, each of the second grouping of arcuate articulating members extending outwardly from the second central member.

Alternatively or additionally, the second central member may be coaxial with the first central member.

Alternatively or additionally, the first grouping of arcuate articulating members may curve in a first direction and the second grouping of arcuate articulating members may curve in a second direction that is opposite the first direction.

Alternatively or additionally, the first grouping of arcuate articulating members and the second grouping of arcuate articulating members may curve in the same direction.

Alternatively or additionally, the first grouping of arcuate articulating members and the second grouping of arcuate articulating members may curve in the same direction but are rotationally offset.

Alternatively or additionally, the expandable frame may further include a third central member, and a third grouping of arcuate articulating members joined at one end to the second central member, each of the third plurality of arcuate articulating members extending outwardly from the third central member.

Alternatively or additionally, the implantable medical device may include a plurality of anchor members secured relative to at least some of the first grouping of arcuate articulating members.

Alternatively or additionally, the implantable medical device may include an LAAC (left atrial appendage closure) device.

Another example may be found in a left atrial appendage closure (LAAC) device. The LAAC device includes an expandable frame moveable between a collapsed configuration for delivery and an expanded configuration for deployment, the expandable frame having a variable diameter in the expanded configuration. The expandable frame includes a central member and a grouping of arcuate articulating members joined at one end to the central member, each of the grouping of arcuate articulating members extending outwardly from the central member. The LAAC device includes a covering disposed relative to the grouping of arcuate articulating members.

Alternatively or additionally, the expandable frame may be biased into the collapsed configuration for delivery, and may be moved into the expanded configuration for deployment by rotating the first central member.

Alternatively or additionally, the expandable frame may be biased into the expanded configuration for deployment, and may be moved into the expanded configuration by removing a constraint that holds the expandable frame in the collapsed configuration for delivery.

Another example may be found in a left atrial appendage closure (LAAC) device. The LAAC device includes an expandable frame moveable between a collapsed configuration for delivery and an expanded configuration for deployment, the expandable frame having a variable diameter in the expanded configuration. The expandable frame includes a first central member, a first grouping of arcuate articulating members joined at one end to the first central member, each of the first grouping of arcuate articulating members extending outwardly from the first central member, a second central member, and a second grouping of arcuate articulating members joined at one end to the second central member, each of the second grouping of arcuate articulating members extending outwardly from the second central member. The LAAC device includes a covering disposed relative to one of the groupings of arcuate articulating members.

Alternatively or additionally, the first grouping of arcuate articulating members may curve in a first direction and the second grouping of arcuate articulating members may curve in a second direction that is opposite the first direction.

Alternatively or additionally, the first grouping of arcuate articulating members and the second grouping of arcuate articulating members may curve in the same direction.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a partial cross-sectional view of an LAA (left atrial appendage);

FIG. 2 is a perspective view of an illustrative LAAC device;

FIG. 3 is a perspective view of an illustrative expandable frame that may be used within an LAAC device, shown in an expanded configuration;

FIG. 4 is a perspective view of the illustrative expandable frame of FIG. 3, shown in a collapsed configuration;

FIG. 5 is a perspective view of an illustrative expandable frame that may be used within an LAAC device, shown in an expanded configuration;

FIG. 6 is a perspective view of an illustrative expandable frame that may be used within an LAAC device, shown in an expanded configuration;

FIG. 7 is a perspective view of an illustrative expandable frame that may be used within an LAAC device, shown in an expanded configuration;

FIG. 8 is a perspective view of an illustrative expandable frame that may be used within an LAAC device, shown in an expanded configuration;

FIG. 9 is a perspective view of the illustrative expandable frame of FIG. 8, shown in a collapsed configuration; and

FIGS. 10A, 10B and 10C together provide views of an illustrative membrane material that may be used as a covering on the LAAC devices described herein.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the present disclosure. Those skilled in the art will recognize that the various elements described and/or shown may be arranged in various combinations and configurations without departing from the scope of the disclosure. The detailed description and drawings illustrate example embodiments of the disclosure. However, in the interest of clarity and ease of understanding, while every feature and/or element may not be shown in each drawing, the feature(s) and/or element(s) may be understood to be present regardless, unless otherwise specified.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For simplicity and clarity purposes, not all elements of the present disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned in an effort to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The terms “monolithic” and “unitary” shall generally refer to an element or elements made from or consisting of a single structure or base unit/element. A monolithic and/or unitary element shall exclude structure and/or features made by assembling or otherwise joining multiple discrete elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to use the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The following figures illustrate selected components and/or arrangements of an implant for occluding the left atrial appendage, a system for occluding the left atrial appendage, and/or methods of using the implant and/or the system. It should be noted that in any given figure, some features may not be shown, or may be shown schematically, for simplicity. Additional details regarding some of the components of the implant and/or the system may be illustrated in other figures in greater detail. While discussed in the context of occluding the left atrial appendage, the implant and/or the system may also be used for other interventions and/or percutaneous medical procedures within a patient. Similarly, the devices and methods described herein with respect to percutaneous deployment may be used in other types of surgical procedures, as appropriate. For example, in some examples, the devices may be used in a non-percutaneous procedure. Devices and methods in accordance with the disclosure may also be adapted and configured for other uses within the anatomy.

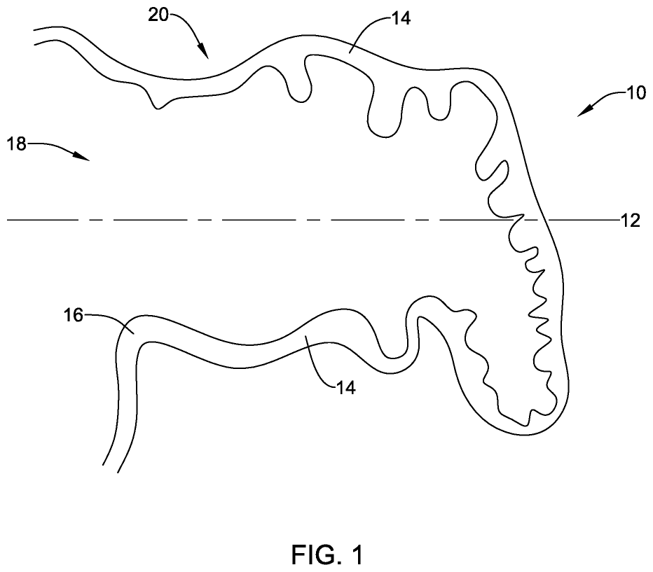

FIG. 1 is a partial cross-sectional view of a left atrial appendage 10. In some embodiments, the left atrial appendage (LAA) 10 may have a complex geometry and/or irregular surface area. It will be appreciated that the illustrated LAA 10 is merely one of many possible shapes and sizes for the LAA 10, which may vary from patient to patient. In some cases, the LAA 10 may be substantially shallower than the illustrated LAA 10. In some cases, the LAA 10 may be shallower than the illustrated LAA 10, and may additionally include a proximal lobe, for example. Those of skill in the art will also recognize that the medical devices, systems, and/or methods disclosed herein may be adapted for various sizes and shapes of the LAA 10, as necessary. The left atrial appendage 10 may include a generally longitudinal axis 12 arranged along a depth of a main body 20 of the left atrial appendage 10. The main body 20 may include a lateral wall 14 and an ostium 16 forming a proximal mouth 18. In some examples, a lateral extent of the ostium 16 and/or the lateral wall 14 may be smaller or less than a depth of the main body 20 along the longitudinal axis 12, or a depth of the main body 20 may be greater than a lateral extent of the ostium 16 and/or the lateral wall 14. In some examples, the LAA 10 may narrow quickly along the depth of the main body 20 or the left atrial appendage may maintain a generally constant lateral extent along a majority of depth of the main body 20. In some examples, the LAA 10 may include a distalmost region formed or arranged as a tail-like element associated with a distal portion of the main body 20. In some examples, the distalmost region may protrude radially or laterally away from the longitudinal axis 12.

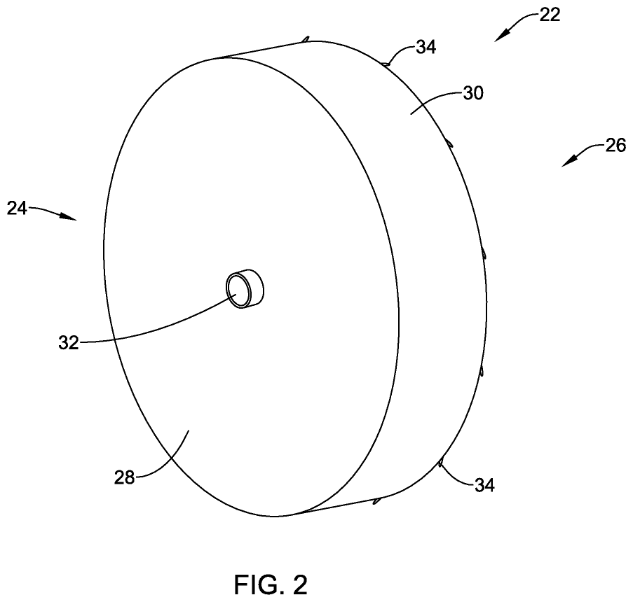

In some instances, a device known as an LAAC (left atrial appendage closure) device may be implanted within the LAA 10, such as near or within the ostium 16, in order to seal off the interior of the LAA 10 from the rest of the heart interior. FIG. 2 is a perspective view of an illustrative LAAC 22 that may be implanted within the LAA 10, such as near or within the ostium 16, in order to seal off the interior of the LAA 10 from the rest of the heart interior.

The LAAC 22 may be considered as having a proximal-facing side 24 and a distal-facing side 26. When implanted, the proximal-facing side 24 will face away from the LAA 10 and the distal-facing side 26 will face into the LAA 10. In some cases, the LAAC 22 includes a covering 28 that covers or at least substantially covers the proximal-facing side 24. In some cases, the covering 28 may extend at least partially over a side 30 of the LAAC 22.

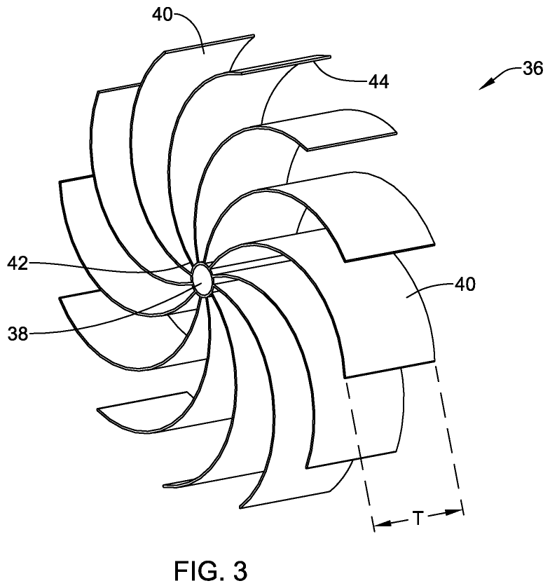

While not expressly shown in FIG. 2, the LAAC 22 includes an expandable frame that is adapted to move between a collapsed configuration for delivery and an expanded configuration (as shown) for deployment. FIGS. 3 through 9 provide illustrative but non-limiting examples of expandable frames that may be used in the LAAC 22. While the expandable frame in the LAAC 22 is not visible in FIG. 2, several features of the expandable frame are. The LAAC 22 includes a mounting feature 32 by which the LAAC 22 may be releasably securable to a delivery device for delivering the LAAC 22. In some cases, the mounting feature 32 may provide a threaded engagement with a delivery device. Other attachment mechanisms are contemplated. the LAAC 22 also includes anchor features 34 that in some cases are equidistantly spaced about a periphery of the distal-facing side 26. The anchor features 34 may help to secure the LAAC 22 in place.

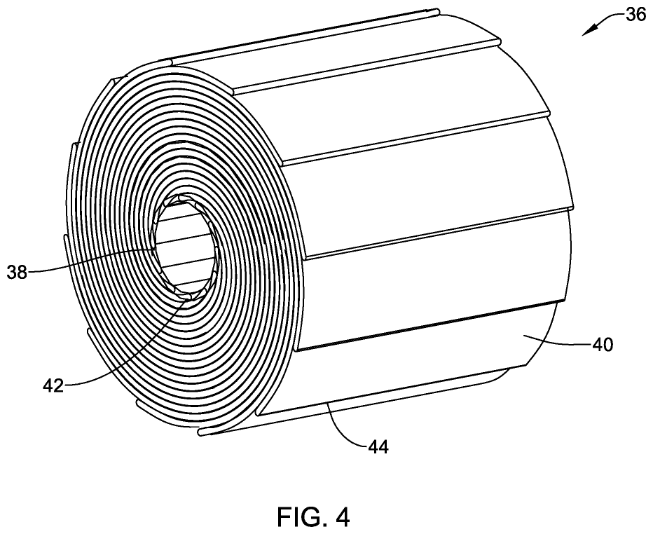

FIG. 3 is a perspective view of an illustrative expandable frame 36 that may be used forming the LAAC 22. In FIG. 3, the expandable frame 36 is shown in an expanded configuration. FIG. 4 provides a corresponding collapsed configuration for the expandable frame 36. In some cases, the expandable frame 36 may be biased into the expanded configuration, and may be temporarily constrained into the collapsed configuration during delivery. Once the constraint is removed, the expandable frame 36 may be free to regain its expanded configuration, or at least as much of its expanded configuration allowed by the tissue of the LAA 10 in which the LAAC 22 is deployed.

While the expandable frame 36 is pictured in FIG. 3 as symmetrical, it will be appreciated that this is an idealization. The expandable frame 36 includes a central member 38 and a plurality of arcuate articulating members 40 that have a first end 42 secured to the central member 38 and an opposing second end 44. In some cases, some of the arcuate articulating members 40 may expand to a further extent than others of the arcuate articulating members 40 as a result of tissue interference.

In some cases, the expandable frame 36 may be biased into the collapsed configuration, and may be actuated into the expanded configuration upon deployment. In some cases, for example, the expandable frame 36 may be rotated by rotating the central member 38. Rotation in a first direction may cause the expandable frame 36 to expand, while rotation in an opposing second direction may cause the expandable frame 36 to contract.

While the expandable frame 36 is shown with a total of twelve arcuate articulating members 40, this is just an example. In some cases, the expandable frame 36 may have fewer than twelve arcuate articulating members 40. In some cases, the expandable frame 36 may have more than twelve arcuate articulating members 40. In some cases, each of the arcuate articulating members 40 may have the same width and the same length. In some cases, at least some of the arcuate articulating members 40 may have different widths and/or different lengths. An expandable frame 36 intended for implantation as part of the LAAC 22 into a larger LAA 10 may have additional arcuate articulating members 40, or may have longer arcuate articulating members 40. An expandable frame 36 intended for implantation as part of the LAAC 22 into a smaller LAA 10 may have fewer arcuate articulating members 40, or may have shorter arcuate articulating members 40, for example.

In some cases, the expandable frame 36 has an overall thickness “T” that is in a range of about 3 to 8 millimeters (mm) or about 6 to 8 mm. This allows for creation of an LAA utilizing the expandable frame 36 that will fit into a shallow LAA 10, or perhaps an LAA 10 that includes a proximal lobe. Each of the arcuate articulating members 40 may be formed of a shape memory material such as Nitinol. Each of the arcuate articulating members 40 may be formed of spring steel. In some cases, each of the arcuate articulating members 40 may be formed of a shape memory polymer or a combination or composite of shape memory polymer/metal material such as a polymer base material and a Nitinol wire or frame to form the petal member. Each of the arcuate articulating members 40 may have a thickness that is in a range of about 0.0005 inches to about 0.012 inches.

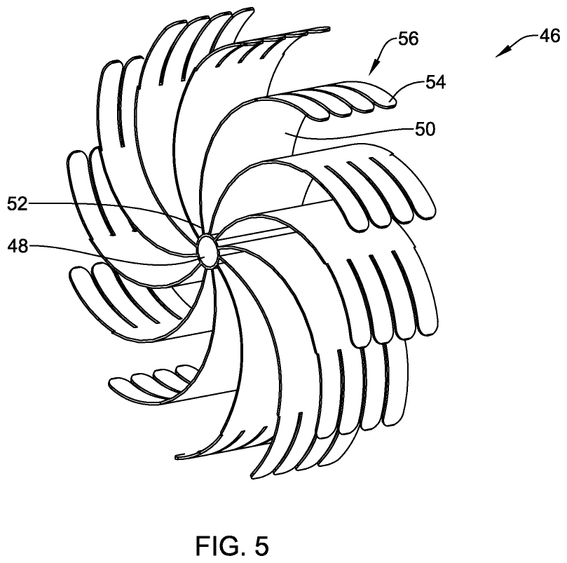

FIG. 5 is a perspective view of an illustrative expandable frame 46 that may be used forming the LAAC 22. In FIG. 5, the expandable frame 46 is shown in an expanded configuration. While no corresponding Figure shows the expandable frame 46 in a collapsed configuration, it will be appreciated that the view in FIG. 3 of the collapsed expandable frame 36 provides a general idea of the collapsed configuration of the expandable frame 46.

In some cases, the expandable frame 46 may be biased into the expanded configuration, and may be temporarily constrained into the collapsed configuration during delivery. Once the constraint is removed, the expandable frame 46 may be free to regain its expanded configuration, or at least as much of its expanded configuration allowed by the tissue of the LAA 10 in which the LAAC 22 is deployed.

While the expandable frame 46 is pictured in FIG. 5 as symmetrical, it will be appreciated that this is an idealization. The expandable frame 46 includes a central member 48 and a plurality of arcuate articulating members 50 that have a first end 52 secured to the central member 48 and an opposing second end 54. In some cases, as shown, the second end 54 of each of the arcuate articulating members 50 may include one or more fingers 56 that help the expandable frame 46 to better adjust to tissue anatomy variability in size and shape, and to help enhance securement of the expandable frame 46. In some cases, some of the arcuate articulating members 62 may expand to a further extent than others of the arcuate articulating members 62 as a result of tissue interference.

In some cases, the expandable frame 46 may be biased into the collapsed configuration, and may be actuated into the expanded configuration upon deployment. In some cases, for example, the expandable frame 46 may be rotated by rotating the central member 48. Rotation in a first direction may cause the expandable frame 46 to expand, while rotation in an opposing second direction may cause the expandable frame 46 to contract.

While the expandable frame 46 is shown with a total of twelve arcuate articulating members 50, this is just an example. In some cases, the expandable frame 46 may have fewer than twelve arcuate articulating members 50. In some cases, the expandable frame 46 may have more than twelve arcuate articulating members 50. In some cases, each of the arcuate articulating members 50 may have the same width and the same length. In some cases, at least some of the arcuate articulating members 50 may have different widths and/or different lengths. Some of the arcuate articulating members 50 may include fingers 54 and others may not, for example.

An expandable frame 46 intended for implantation as part of the LAAC 22 into a larger LAA 10 may have additional arcuate articulating members 50, or may have longer arcuate articulating members 50. An expandable frame 46 intended for implantation as part of the LAAC 22 into a smaller LAA 10 may have fewer arcuate articulating members 50, or may have shorter arcuate articulating members 50, for example.

In some cases, the expandable frame 46 has an overall thickness “T” that is in a range of about 3 to 8 millimeters (mm) or about 6 to 8 mm. This allows for creation of an LAA utilizing the expandable frame 46 that will fit into a shallow LAA 10, or perhaps an LAA 10 that includes a proximal lobe. Each of the arcuate articulating members 50 may be formed of a shape memory material such as Nitinol. Each of the arcuate articulating members 50 may be formed of spring steel. In some cases, each of the arcuate articulating members 50 may be formed of a shape memory polymer or a combination or composite of shape memory polymer/metal material such as a polymer base material and a Nitinol wire or frame to form the petal member. Each of the arcuate articulating members 50 may have a thickness that is in a range of about 0.0005 inches to about 0.012 inches.

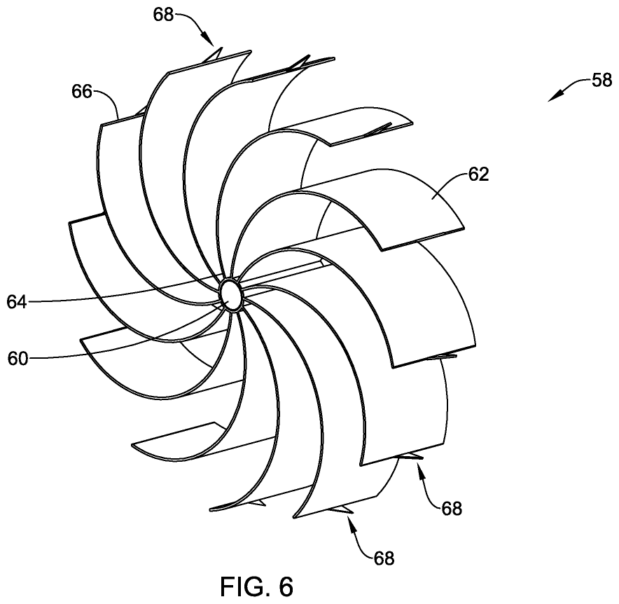

FIG. 6 is a perspective view of an illustrative expandable frame 58 that may be used forming the LAAC 22. In FIG. 6, the expandable frame 58 is shown in an expanded configuration. While no corresponding Figure shows the expandable frame 58 in a collapsed configuration, it will be appreciated that the view in FIG. 3 of the collapsed expandable frame 36 provides a general idea of the collapsed configuration of the expandable frame 58.

In some cases, the expandable frame 58 may be biased into the expanded configuration, and may be temporarily constrained into the collapsed configuration during delivery. Once the constraint is removed, the expandable frame 58 may be free to regain its expanded configuration, or at least as much of its expanded configuration allowed by the tissue of the LAA 10 in which the LAAC 22 is deployed.

While the expandable frame 58 is pictured in FIG. 6 as symmetrical, it will be appreciated that this is an idealization. The expandable frame 58 includes a central member 60 and a plurality of arcuate articulating members 62 that have a first end 64 secured to the central member 60 and an opposing second end 66. In some cases, as shown, the second end 66 of each of the arcuate articulating members 62 may include an anchor feature 68, which can help to secure the expandable frame 58 in place relative to the LAA 10. In some cases, some of the arcuate articulating members 62 may expand to a further extent than others of the arcuate articulating members 62 as a result of tissue interference.

In some cases, the expandable frame 58 may be biased into the collapsed configuration, and may be actuated into the expanded configuration upon deployment. In some cases, for example, the expandable frame 58 may be rotated by rotating the central member 60. Rotation in a first direction may cause the expandable frame 58 to expand, while rotation in an opposing second direction may cause the expandable frame 58 to contract.

While the expandable frame 58 is shown with a total of twelve arcuate articulating members 62, this is just an example. In some cases, the expandable frame 58 may have fewer than twelve arcuate articulating members 62. In some cases, the expandable frame 58 may have more than twelve arcuate articulating members 62. In some cases, each of the arcuate articulating members 62 may have the same width and the same length. In some cases, at least some of the arcuate articulating members 62 may have different widths and/or different lengths. Some of the arcuate articulating members 62 may include anchor features 68 and others may not, for example.

An expandable frame 58 intended for implantation as part of the LAAC 22 into a larger LAA 10 may have additional arcuate articulating members 62, or may have longer arcuate articulating members 62. An expandable frame 58 intended for implantation as part of the LAAC 22 into a smaller LAA 10 may have fewer arcuate articulating members 62, or may have shorter arcuate articulating members 62, for example.

In some cases, the expandable frame 58 has an overall thickness “T” that is in a range of about 3 to 8 millimeters (mm) or about 6 to 8 mm. This allows for creation of an LAA utilizing the expandable frame 46 that will fit into a shallow LAA 10, or perhaps an LAA 10 that includes a proximal lobe. Each of the arcuate articulating members 62 may be formed of a shape memory material such as Nitinol. Each of the arcuate articulating members 62 may be formed of spring steel. Each of the arcuate articulating members 62 may have a thickness that is in a range of about 0.0005 inches to about 0.012 inches.

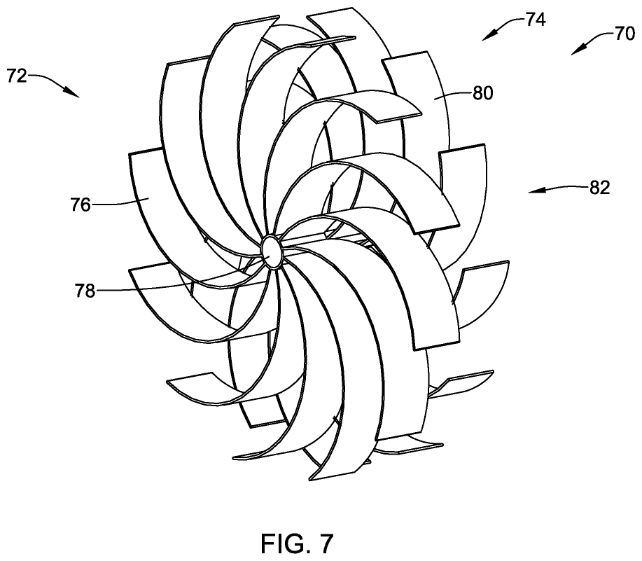

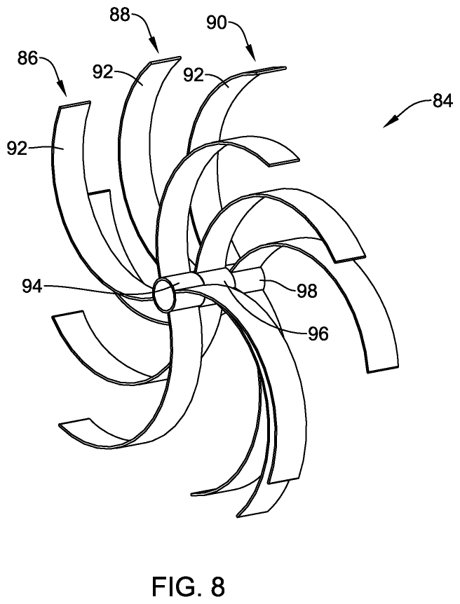

FIG. 7 is a perspective view of an illustrative expandable frame 70 that may be used forming the LAAC 22. In FIG. 7, the expandable frame 70 is shown in an expanded configuration. The expandable frame 70 includes a first grouping 72 of arcuate articulating members and a second grouping 74 of arcuate articulating members. As shown, the first grouping 72 of arcuate articulating members curve in a first direction while the second grouping 74 of arcuate articulating members curve in a second direction that opposes the first direction. In some cases, the first grouping 72 of arcuate articulating members and the second grouping 74 of arcuate articulating members may each curve in the same direction, but may be rotationally offset from each other (as will be discussed with respect to FIG. 8).

In some cases, the expandable frame 70 may be biased into the expanded configuration, and may be temporarily constrained into the 70 configuration during delivery. Once the constraint is removed, the expandable frame 70 may be free to regain its expanded configuration, or at least as much of its expanded configuration allowed by the tissue of the LAA 10 in which the LAAC 22 is deployed.

While the expandable frame 70 is pictured in FIG. 7 as symmetrical, it will be appreciated that this is an idealization. The first grouping 72 of arcuate articulating members includes a number of arcuate articulating members 76 that extend outwardly from a first central member 78 and the second grouping 74 of arcuate articulating members includes a number of arcuate articulating members 80 that extend outwardly from a second central member 82 (not visible in this view). In some cases, some of the arcuate articulating members 76 and/or some of the arcuate articulating members 80 may expand to a further extent than others as a result of tissue interference.

While the expandable frame 70 is shown with a total of twelve arcuate articulating members 76 and a total of twelve arcuate articulating members 80, this is just an example. In some cases, the expandable frame 70 may have fewer than twelve arcuate articulating members 76 and/or 80. In some cases, the expandable frame 70 may have more than twelve arcuate articulating members 76 and/or 80. In some cases, each of the arcuate articulating members 76 and/or 80 may have the same width and the same length. In some cases, at least some of the arcuate articulating members 76 and/or 80 may have different widths and/or different lengths. Some of the arcuate articulating members 76 and/or 78 may include anchor features and others may not, for example.

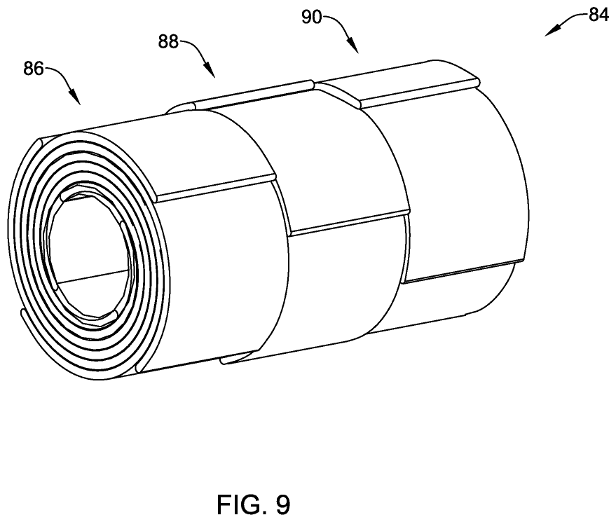

FIG. 8 is a perspective view of an illustrative expandable frame 84 that may be used forming the LAAC 22. FIG. 8 shows the expandable frame 84 in an expanded configuration and FIG. 9 shows the expandable frame 84 in a collapsed configuration. The expandable frame 84 includes a first grouping 86 of arcuate articulating members, a second grouping 88 of arcuate articulating members and a third grouping 90 of arcuate articulating members. Each of the first grouping 86, the second grouping 88 and the third grouping 90 each curve in the same direction, but are rotationally offset from each other.

In some cases, the expandable frame 84 may be biased into the expanded configuration, and may be temporarily constrained into the 84 configuration during delivery. Once the constraint is removed, the expandable frame 84 may be free to regain its expanded configuration, or at least as much of its expanded configuration allowed by the tissue of the LAA 10 in which the LAAC 22 is deployed.

While the expandable frame 84 is pictured in FIG. 8 as symmetrical, it will be appreciated that this is an idealization. The first grouping 86 of arcuate articulating members includes a number of arcuate articulating members 92 extending outwardly from a first central member 78 and the second grouping 74 of arcuate articulating members includes a number of arcuate articulating members 80 that extend outwardly from a second central member 82 (not visible in this view). In some cases, some of the arcuate articulating members 76 and/or some of the arcuate articulating members 80 may expand to a further extent than others as a result of tissue interference.

While the expandable frame 70 is shown with a total of six arcuate articulating members 92 in each of the first grouping 86, the second grouping 88 and the third grouping 90, this is just an example. Some of the first grouping 86, the second grouping 88 and the third grouping 90 may have additional arcuate articulating members 92, or fewer. Some of the arcuate articulating members 92 may be shorter or longer, or have varying widths. Some of the arcuate articulating members 92 may include anchor features and others may not, for example.

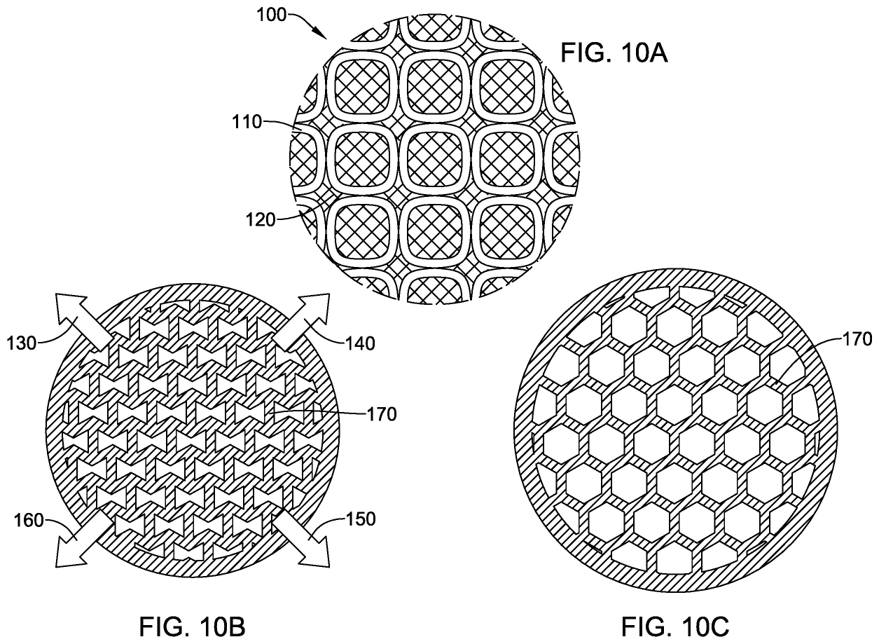

As will be appreciated, in some cases the covering (such as the covering 28 shown in FIG. 2) may have to accommodate changes in the dimensions of the expandable frame. In other words, the covering may have to be able to stretch. FIGS. 10A, 10B and 10C together provide details of a covering 100 that may be used with the LAAC devices described herein. The covering 100 includes a webbing 110 that is made from a relatively thick fiber. A webbing 120 spans the distance between the relatively thick fibers forming the webbing 110 and is formed from relatively thin fibers. In some cases, the webbing 110 may be formed of fibers having an average diameter in a range of 5 to 10 μm and the webbing 120 may be formed of fibers having an average diameter in a range of 25 to 100 μm. In some cases, the webbing 110 is laid out in a honeycomb fashion, but this is not required in all cases.

In some cases, the webbing 110 may be formed of fibers that have a relatively large fraction of elastomer and a relatively smaller fraction of a second polymer such as but not limited to PET (polyethylene terephthalate). In some cases, the webbing 110 may be formed of fibers that are at least 50 percent elastomer and the webbing 120 may be formed of fibers that are at least 50 percent PET. In some cases, the webbing 110 may be formed of fibers that include about 70 percent elastomer and about 30 percent PET. In some cases, the webbing 120 may be formed of fibers that have a relatively large fraction of PET and a relatively smaller fraction of an elastomer. In some cases, the webbing 120 may be formed of fibers that include about 30 percent PET and about 70 percent elastomer. The elastomers used in the webbing 110 and the webbing 120 may include one or more of fluoroelastomers, polyurethane elastomers, Pebax, thermoplastic elastomers, copolyester elastomer, hydrophilic elastomers, polyamide 11 or polyether segments.

FIGS. 10B and 10C together illustrate how the covering 100 responds to applied forces. In particular, FIGS. 10B and 10C together show that tension applied in any direction, as indicated by the arrows 130, 140, 150 and 160, result in equal porosity. In FIG. 10C, the mesh 170 can be seen as having equal pore sizes. In some cases, the covering 600 may be considered as exhibiting auxetic properties. In some cases, the covering 100 may include materials such as urethane or nylon. In some cases, the covering 100 may also include radiopaque elements. In some cases, the covering 100 may be a fabric matrix that is formed in an auxetic pattern, such that stretching and compliance in a planar radial axis is equalized and distributed consistently regarding porosity of hemodynamic flow as well as hemostasis.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-clastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super-elastic nitinol in that the linear elastic and/or non-super-clastic nitinol does not display a substantial “superelastic plateau” or “flag region” in its stress/strain curve like super-elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super-elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear clastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, the devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

A sheath or covering (not shown) may be disposed over portions or all of the devices described herein in order to define a generally smooth outer surface. In other embodiments, however, such a sheath or covering may be absent. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An implantable medical device, comprising:
an expandable frame moveable between a collapsed configuration for delivery and an expanded configuration for deployment, the expandable frame having a variable diameter in the expanded configuration, the expandable frame comprising:
a first central member; and
a first grouping of arcuate articulating members joined at one end to the first central member, each of the first grouping of arcuate articulating members extending outwardly from the first central member; and
a covering disposed relative to the first grouping of arcuate articulating members.

2. The implantable medical device of claim 1, wherein the expandable frame is biased into the collapsed configuration for delivery, and is moved into the expanded configuration for deployment by rotating the first central member.

3. The implantable medical device of claim 1, wherein the expandable frame is biased into the expanded configuration for deployment, and is moved into the expanded configuration by removing a constraint that holds the expandable frame in the collapsed configuration for delivery.

4. The implantable medical device of claim 1, wherein the covering extends over the first grouping of arcuate articulating members.

5. The implantable medical device of claim 1, wherein the first grouping of arcuate articulating members is formed of a shape memory material.

6. The implantable medical device of claim 5, wherein the first grouping of arcuate articulating members is adapted to have a remembered shape corresponding to the expanded configuration of the expandable frame.

7. The implantable medical device of claim 1, wherein the expandable frame further comprises:
a second central member; and
a second grouping of arcuate articulating members joined at one end to the second central member, each of the second grouping of arcuate articulating members extending outwardly from the second central member.

8. The implantable medical device of claim 7, wherein the second central member is coaxial with the first central member.

9. The implantable medical device of claim 7, wherein the first grouping of arcuate articulating members curve in a first direction and the second grouping of arcuate articulating members curve in a second direction that is opposite the first direction.

10. The implantable medical device of claim 7, wherein the first grouping of arcuate articulating members and the second grouping of arcuate articulating members curve in the same direction.

11. The implantable medical device of claim 7, wherein the first grouping of arcuate articulating members and the second grouping of arcuate articulating members curve in the same direction but are rotationally offset.

12. The implantable medical device of claim 7, wherein the expandable frame further comprises:
a third central member; and
a third grouping of arcuate articulating members joined at one end to the second central member, each of the third plurality of arcuate articulating members extending outwardly from the third central member.

13. The implantable medical device of claim 1, comprising a plurality of anchor members secured relative to at least some of the first grouping of arcuate articulating members.

14. The implantable medical device of claim 1, comprising an LAAC (left atrial appendage closure) device.

15. A left atrial appendage closure (LAAC) device, comprising:
an expandable frame moveable between a collapsed configuration for delivery and an expanded configuration for deployment, the expandable frame having a variable diameter in the expanded configuration, the expandable frame comprising:
a central member; and
a grouping of arcuate articulating members joined at one end to the central member, each of the grouping of arcuate articulating members extending outwardly from the central member; and
a covering disposed relative to the grouping of arcuate articulating members.

16. The LAAC device of claim 15, wherein the expandable frame is biased into the collapsed configuration for delivery, and is moved into the expanded configuration for deployment by rotating the first central member.

17. The LAAC device of claim 15, wherein the expandable frame is biased into the expanded configuration for deployment, and is moved into the expanded configuration by removing a constraint that holds the expandable frame in the collapsed configuration for delivery.

18. A left atrial appendage closure (LAAC) device, comprising:
an expandable frame moveable between a collapsed configuration for delivery and an expanded configuration for deployment, the expandable frame having a variable diameter in the expanded configuration, the expandable frame comprising:
a first central member;
a first grouping of arcuate articulating members joined at one end to the first central member, each of the first grouping of arcuate articulating members extending outwardly from the first central member;
a second central member;
a second grouping of arcuate articulating members joined at one end to the second central member, each of the second grouping of arcuate articulating members extending outwardly from the second central member; and
a covering disposed relative to the grouping of arcuate articulating members.

19. The LAAC device of claim 18, wherein the first grouping of arcuate articulating members curve in a first direction and the second grouping of arcuate articulating members curve in a second direction that is opposite the first direction.

20. The LAAC device of claim 18, wherein the first grouping of arcuate articulating members and the second grouping of arcuate articulating members curve in the same direction.
